(19) United States
(12) Patent Application Publication
(10) Pub. No.: US 2008/0154148 A1
(43) Pub. Date: Jun. 26, 2008

(54) METHOD AND APPARATUS FOR OPERATING TERMINAL BY USING BRAIN WAVES

(75) Inventors: Beom-Su Chung, Yongin-si (KR); Seong-Joon Jeon, Yongin-si (KR)

Correspondence Address: THE FARRELL LAW FIRM, P.C., 333 EARLE OVINGTON BOULEVARD, SUITE 701, UNIONDALE, NY, 11553, US

(73) Assignee: SAMSUNG ELECTRONICS CO., LTD., Suwon-si (KR)

(21) Appl. No.: 11/961,207

(22) Filed: Dec. 20, 2007

(30) Foreign Application Priority Data
Dec. 20, 2006 (KR) 130938/2006

(51) Int. Cl. A61B 5/0476; G06F 17/30

(52) U.S. Cl. 600/544; 707/100; 707/3; 707/E17.005

(57) ABSTRACT

Disclosed are a method and an apparatus for operating a terminal by using brain waves. The method includes receiving a current brain wave of a terminal user; finding the existence of data matched to the received current brain wave among previously constructed database having reduced data sizes of brain waves of the terminal user and corresponding to at least one codeword; and operating the terminal according to a codeword corresponding to the matched brain wave data when the matched data is found. The apparatus includes a memory for storing previously constructed database having reduced data sizes of brain waves of a terminal user and corresponding to at least one codeword; a data processing unit for processing a current brain wave of the terminal user; a data search unit for finding data matched to the processed current brain wave; and a control unit for operating the terminal according to a codeword corresponding to the matched data.

### PRIORITY

This application claims priority under 35 U.S.C. § 119(a) to an application entitled “Method and Apparatus for Operating Terminal by using Brain Waves” filed in the Korean Intellectual Property Office on Dec. 20, 2006 and assigned Serial No. 2006-130938, the contents of which are incorporated herein by reference.

### BACKGROUND OF THE INVENTION

1. Field of the Invention

The present invention relates to a method and an apparatus for operating a terminal, and in particular, to a method and an apparatus for operating a terminal by using brain waves.

2. Description of the Related Art

The term ‘brain waves’ refers to electrical waves generated from the brain. A brief description will be made of characteristics of brain waves which have generally been known as follows. The types of brain waves generated from the human brain are largely classified into Alpha waves, Beta waves, Theta waves, and Delta waves in terms of frequencies.

The Alpha waves correspond to brain waves generated when we close our eyes and then relax our body, and have a frequency range of 8 to 13 Hz. The Beta waves correspond to most of the brain waves generated when our consciousness is awake, and have a frequency range of 14 to 100 Hz, which is higher than the Alpha waves. The Theta waves are generated in a light sleep state, have a frequency range of 4 to 8 Hz which is even lower than that of the Alpha waves, and are produced in a boundary state between consciousness and dream. The Delta waves have a frequency range that is equal to or lower than 4 Hz, which is much lower than the frequency range of the Theta waves, and correspond to brain waves which are measured the most frequently in a sleeping state or in an unconscious state.

Also, characteristics of brain waves have been known through research on specific patterns of the brain waves generated in different situations, such as when a human being responds to an external stimulus, or when they think in a certain way.

FIG. 1 is a view illustrating the measurement and recording of brain waves generated in response to visual stimuli. In FIG. 1, an S1-match corresponds to brain waves in a case of showing a specific picture to an experiment participant again following the elapse of a prescribed time interval after showing the specific picture to the experiment participant, and an S2-match corresponds to brain waves in a case other than this. It can be found that the S1-match and the S2-match show different waveforms from each other. This result indicates that specific brain waves are generated while thinking or viewing a specified image.

Until now, the arts using brain waves analyze brain waves to put a person's mind in a stable state, or enable brain waves to be generated in order to improve a person's power of concentration and then apply stimuli to the brain, or other parts of the body. The prior art using brain waves has remained in an elementary level, such as simply analyzing brain waves generated from the human brain, and the like.

Also, up to now, in the case of measuring brain waves to analyze brain waves in the field of medical services, huge amounts of data have been needed. Since brain waves are generated in each part of the brain, the more analysis there is of brain waves, the higher the accuracy of brain wave analysis. Therefore, for even more accurate analysis of brain waves, much larger volume of data of brain waves is preferable. Meanwhile, when household appliances, a mobile terminal, and the like are enabled to operate by using brain waves, the convenience of a user can be greatly improved. Nevertheless, since resource capacity for data processing is limited in a mobile terminal, and the like, there is a limit in using brain wave data.

### SUMMARY OF THE INVENTION

Accordingly, the present invention has been made to solve the above-stated problems occurring in the prior art, and the present invention provides a method and an apparatus for operating a terminal by using brain waves.

In accordance with an aspect of the present invention, there is provided a method for operating a terminal, including receiving a current brain wave of a user of the terminal; finding an existence of data matched to the received current brain wave among a previously constructed database having reduced data sizes of brain waves of the user of the terminal and corresponding to at least one codeword; and operating the terminal according to a codeword corresponding to the brain wave matched data when the matched data is found.

In finding the existence of data, there is a method of constructing the database having reduced data sizes and the method includes computing a first data matrix on the brain waves of the user of the terminal; computing a first covariance matrix on the first data matrix; performing a Discrete Karhunen-Lo'eve Transform (DKLT) on the first data matrix by using the first covariance matrix; determining separation values of the DKLT transformed first data matrix by a prescribed number; extracting respective elements corresponding to the determined separation values among the first covariance matrices and determining a second covariance matrix; performing the DKLT on the first data matrix by using the determined second covariance matrix and determining a second data matrix; and configuring the database by using the second data matrix.

The separation value is determined by the following equation:

$${P}_{m ; a / b}^{r} ( k ) = \frac{{[ {\overline{\tilde{z}}}_{m / a}^{r} ( k ) - {\overline{\tilde{z}}}_{m / b}^{r} ( k ) ]}^{2}}{{\sum }_{i = 1}^{I} {[ {\tilde{z}}_{m / a ; i}^{r} ( k ) - {\overline{\tilde{z}}}_{m / a}^{r} ( k ) ]}^{2} + {\sum }_{i = 1}^{I} {[ {\tilde{z}}_{m / b ; i}^{r} ( k ) - {\overline{\tilde{z}}}_{m / b}^{r} ( k ) ]}^{2}} ,$$

where ‘a’ and ‘b’ represent brain wave data different from each other, ‘m’ represents a serial number corresponding to a brain wave detection part, and ‘r’ represents the number of times brain wave data is detected.

Also, the method of receiving a current brain wave includes computing a first data matrix on the received current brain wave; computing a first covariance matrix on the first data matrix; performing a DKLT on the first data matrix by using the first covariance matrix; determining the separation values of the DKLT transformed first data matrix by a prescribed number; extracting respective elements corresponding to the determined separation values among the first covariance matrices and determining a second covariance matrix; and performing the DKLT on the first data matrix by using the determined second covariance matrix and then determining a second data matrix.

The separation value is determined by the equation given above.

In accordance with another aspect of the present invention, there is provided an apparatus for operating a terminal, including a memory for storing a previously constructed database having reduced data sizes of brain waves of a user of the terminal and corresponding to at least one codeword; a data processing unit for processing a current brain wave of the user of the terminal; a data search unit for finding data matched to the processed current brain wave among the database; and a control unit for operating the terminal according to a codeword corresponding to the brain wave matched data when the matched data is found.

Also, the database stored in the memory is constructed by computing a first data matrix on the brain waves of the user of the terminal; computing a first covariance matrix on the first data matrix; performing the DKLT on the first data matrix by using the first covariance matrix; determining the separation values of the DKLT transformed first data matrix by a prescribed number; extracting respective elements corresponding to the determined separation values among the first covariance matrices and determining a second covariance matrix; performing the DKLT on the first data matrix by using the determined second covariance matrix and determining a second data matrix; and configuring the database by using the second data matrix.

The separation value is determined by the equation given above.

The data processing unit carries out computing a first data matrix on the current brain wave of the user of the terminal; computing a first covariance matrix on the first data matrix; performing the DKLT on the first data matrix by using the first covariance matrix; determining the separation values of the DKLT transformed first data matrix by a prescribed number; extracting respective elements corresponding to the determined separation values among the first covariance matrices and determining a second covariance matrix; performing the DKLT on the first data matrix by using the determined second covariance matrix and determining a second data matrix; and reducing a size of the current brain wave by using the second data matrix.

The separation value is determined by the equation given above.

### BRIEF DESCRIPTION OF THE DRAWINGS

The above and other features, aspects, and advantages of the present invention will become more apparent from the following detailed description when taken in conjunction with the accompanying drawings, in which:

FIG. 1 is a view illustrating the measurement and recording of brain waves generated in response to visual stimuli;

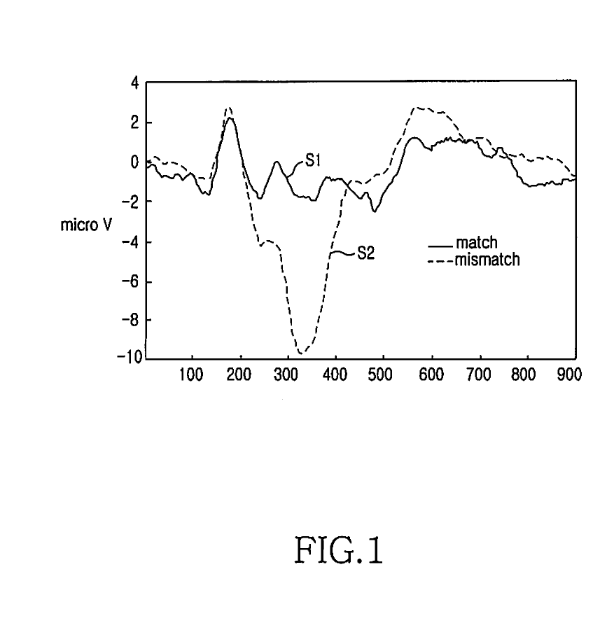

FIG. 2 is a schematic diagram illustrating a process for using brain wave data in a terminal according to the present invention;

FIG. 3 is a flowchart illustrating a procedure for enabling a terminal to operate by using brain waves according to the present invention;

FIG. 4 is a flowchart illustrating a procedure in which a terminal reduces a brain wave data size by using a Discrete Karhunen-Lo'eve Transform (DKLT) according to the present invention; and

FIG. 5 is a block diagram illustrating the configuration of a terminal which operates by using brain wave data according to the present invention.

### DETAILED DESCRIPTION OF THE EXEMPLARY EMBODIMENTS

Hereinafter, exemplary embodiments of the present invention will be described in detail with reference to the accompanying drawings. Further, in the following description of the present invention, a detailed description of known functions and configurations incorporated herein is omitted when it may make the subject matter of the present invention rather unclear.

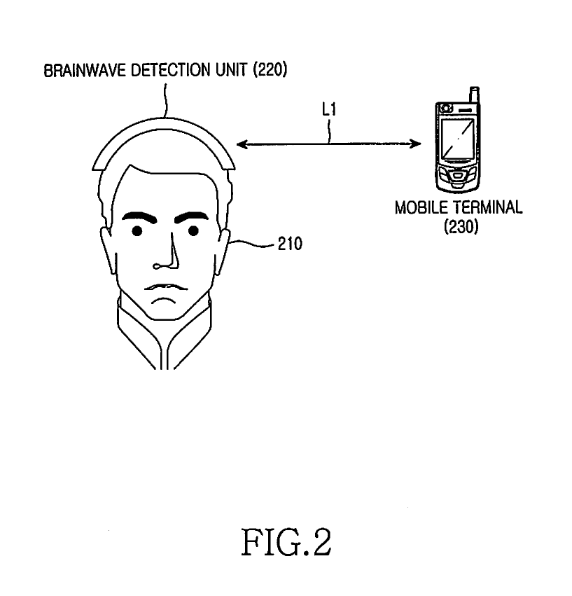

FIG. 2 is a schematic diagram illustrating a process for using brain wave data in a terminal according to the present invention. Brain waves generated from a human brain 210 are received by a terminal 230 through a brain wave detection device 220. The brain wave detection device 220 can be connected to the terminal 230 by a wire connection or by wireless connection L1. The configuration of the brain wave detection device 220 is not included in the present invention, and can be embodied based on various technological configurations. Accordingly, a detailed description of the configuration of the brain wave detection device will be omitted.

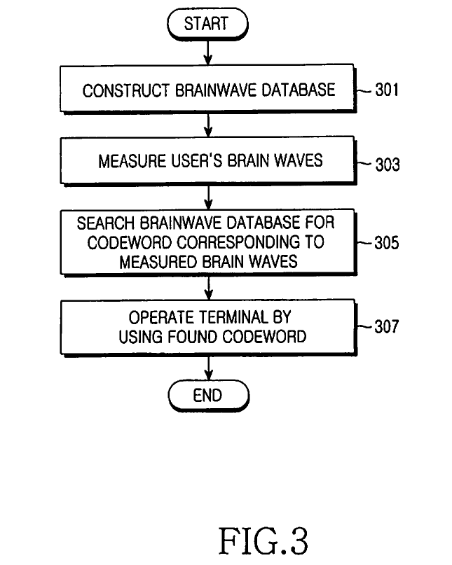

FIG. 3 is a flowchart illustrating a procedure for enabling a terminal to operate by using brain waves according to the present invention. In step 301, brain wave data is constructed as a database. A terminal user's own brain wave data representing a codeword that the user intends to use in the terminal is stored in the terminal by each codeword. The codeword refers to various kinds of instructions which operate the terminal, or various types of characters and numerals that can be entered through the terminal. For example, when it is assumed that ten pieces of brain wave data (Data 1, Data 2 . . . , Data 10) different from one another correspond to brain wave data respectively relevant to numerals 1, 2, 3 . . . , 10, the ten pieces of brain wave data and respective codewords corresponding to the ten pieces of brain wave data, such as {Data 1=1, Data 2=2, . . . Data 10=10}, are stored in the terminal as a database.

In step 303, brain waves are measured through the brain wave detection device 220. Namely, brain waves actually generated from the user are input through the brain wave detection device 220, and are then measured thereby. In step 305, after the database has been constructed, measured brain waves are compared with the database, and brain waves matched to each other are found. The database constructed in step 301 is searched for brain waves matched to the brain waves measured in step 305. In step 305, a filter is used. A representative example of the filter corresponds to a matched filter.

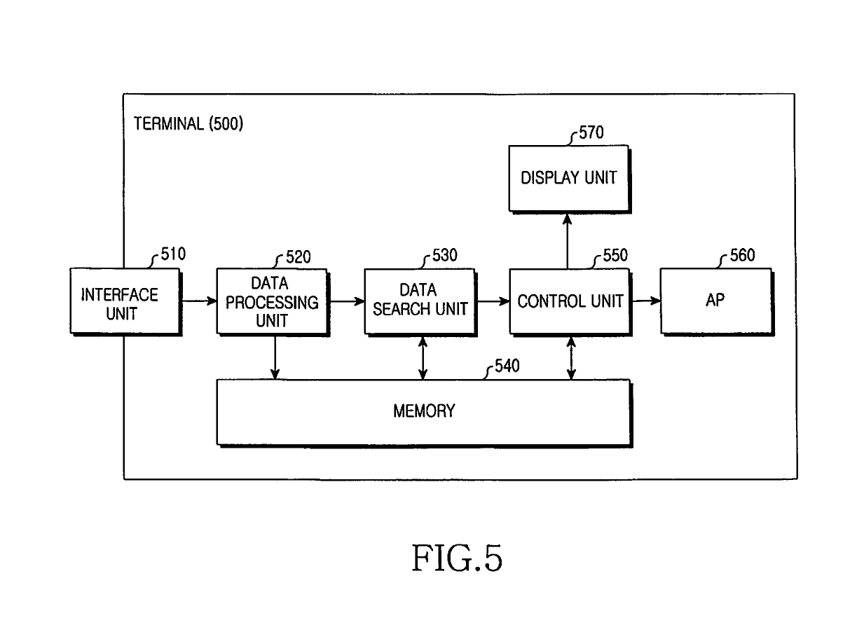

The matched filter corresponds to a filter for passing only a signal that a system designer desires. When a designer can predict a signal that can be input into a certain filter when he or she configures the filter, the designer can configure such a filter that receives only a form of the predicted signal. When a matched filter in regard to brain waves stored in the database is equipped, and when input brain wave data is filtered through the matched filter, brain waves matched to the measured brain waves can be found in the database. It is understood that the matched filter can be replaced with an arbitrary filter having the function capable of searching the database for the presence of brain waves matched to the measured brain waves. An efficient processing may be fulfilled in steps 301 and 305, when the size of brain wave data is small. As described above, this is because the volume of storage capacity is limited in a mobile terminal. Also, as the search speed of the database becomes faster when the size of brain wave data becomes smaller, the performance of an overall system can be improved. Hence, in the present invention, a suggestion will be made of a method for reducing the size of a brain wave database. A description will be made of this method, referring to FIG. 5.

In step 307, the terminal operates according to a codeword found in step 305. For instance, when measured brain waves are matched to Data 1, an abbreviated number corresponding to a codeword ‘1’ can be enabled.

In the terminal, an operation enabled through the above codeword is as follows. For example, a phone call can be made at an abbreviated number corresponding to brain waves. Also, through an operation for enabling a display unit to display a brain wave message, a simple conversation can be held with those around him or her. Moreover, characters corresponding to brain waves can be transferred to another user through Short Message Service (SMS). When the present invention is used for a person with disabilities to operate the terminal, the present invention can be highly useful.

As brain waves used in steps illustrated in FIG. 3, a single brain wave (i.e., a single Electroencephalogram (EEG)) or the average brain wave (i.e., the average EEG) can be selected depending on setting of a user or a designer. The single brain wave refers to using, as brain wave data, measurement values obtained by measuring brain waves once. The average brain wave refers to using, as brain wave data, the average of measurement values obtained by measuring brain waves at least twice.

When the single brain wave is used, the data size of brain waves can be reduced. However, there is such a disadvantage that the accuracy of brain wave analysis can go down. When the average brain wave is used, the data size of brain waves can become large. However, since the effect of noises which can be caused during the measurement of brain waves can be minimized, there is such an advantage that the accuracy of brain wave analysis goes up. The selection of the single brain wave or the average brain wave can be different depending on the property of an application. When the property of a brain wave message can be fully expressed, even by using the single brain wave, the single brain wave can be selected. Namely, when characteristics of brain waves are expressed well, even through one-time measurement of brain waves, it is efficient to use the single brain wave. For example, when user certification for internet banking is performed by using brain waves in a terminal, the single brain wave can be employed. This is because a certain user's brain waves can be easily distinguished from another user's brain waves, even though the single brain wave is employed, for brain waves responding to the same stimulus are revealed very differently according to different users. However, when characteristics of brain waves are not expressed well through one-time measurement of brain waves, the average brain wave has to be used. Therefore, the number of times in brain wave measurement can be determined experimentally in such a manner as to show the best performance as needed by an application. Hereinafter, a description will be made of a procedure for reducing the abovementioned data size of brain waves.

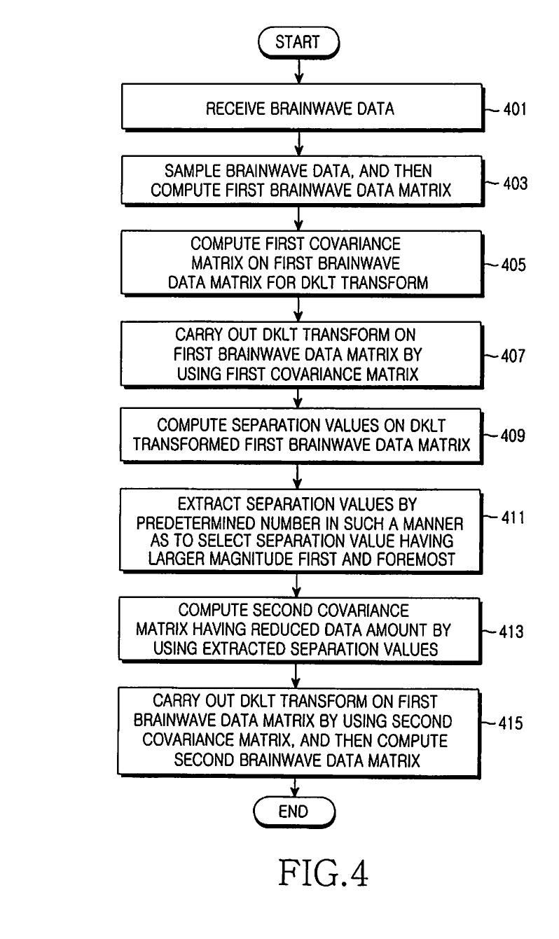

FIG. 4 is a flowchart illustrating a procedure in which a terminal reduces a brain wave data size by using a Discrete Karhunen-Lo'eve Transform (DKLT) according to the present invention. The procedure depicted in FIG. 4 is applied to both step 301 of constructing a database and the step of finding a codeword corresponding to brain waves measured in step 303. The number of codewords respectively corresponding to brain waves can be one or more.

In step 401, the terminal receives brain wave data, and in step 403, it samples the received brain wave data to compute a first brain wave data matrix. In step 405, the terminal computes a first covariance matrix on the first brain wave data matrix to carry out a DKLT. In step 407, the terminal carries out a DKLT on the first brain wave data matrix by using the computed first covariance matrix. In step 409, the terminal computes a separation value on a DKLT transformed first brain wave data matrix. The separation value corresponds to a numerical value used to indicate the extent to which relevant brain waves have such a feature that the relevant brain waves are distinguished from different brain waves. A separation value corresponding to each element of the DKLT transformed first brain wave data matrix can be obtained.

The concept of the above separation value will be exemplified. Let's assume that a situation where a certain teacher expresses bodily features of a number of students to another teacher. The teacher can explain “John wears glasses and has prominent double-edged eyelids.”, and “Mary is a girl who has beautiful golden hair and deep blue eyes.” At this time, the most characteristic parts are the spectacles and prominent double-edged eyelids among John's various bodily features (e.g., eyes, nose, mouth, hair, etc.). In this manner, a part in which a feature of brain waves is best expressed among a piece of measured brain waves is called the separation value. The number of separation values can be different as needed by a system. The more separation values there are (in this case, measured brain waves turn closer to the original brain waves), the higher the degree of recognition on brain waves there is. However, as time required to recognize brain waves or search time of a database increases, the number of separation values can be determined in the manner of trade-off, depending on an application or a system situation.

A method used in a process for computing a separation value, of the present invention, is defined by the following Equation (1):

$${P}_{m ; a / b}^{r} ( k ) = \frac{{[ {\overline{\tilde{z}}}_{m / a}^{r} ( k ) - {\overline{\tilde{z}}}_{m / b}^{r} ( k ) ]}^{2}}{{\sum }_{i = 1}^{I} {[ {\tilde{z}}_{m / a ; i}^{r} ( k ) - {\overline{\tilde{z}}}_{m / a}^{r} ( k ) ]}^{2} + {\sum }_{i = 1}^{I} {[ {\tilde{z}}_{m / b ; i}^{r} ( k ) - {\overline{\tilde{z}}}_{m / b}^{r} ( k ) ]}^{2}} .$$

where ‘a’ and ‘b’ represent brain wave data different from each other, and ‘m’ represents a serial number corresponding to a brain wave detection part. For example, when brain wave detection is performed in three parts, serial numbers 1, 2, and 3 are assigned to the respective detection parts.

‘r’ represents the number of times brain wave data is detected. For example, when brain wave data on a numeral ‘1’ is detected once, the value of ‘r’ equals ‘1’, and when brain wave data on the numeral ‘1’ is detected four times, the value of ‘r’ equals ‘4.’

\overline{\tilde{z}}_{m/a}^{r}(k) represents the k-th element of the average data ‘a’ using ‘r’ number of brain wave data matrices detected at an ‘m’ part.

‘i’ and ‘j’ are applied only to a case using the average brain wave. When the value of ‘r’ equals ‘4’, the values of ‘I’ equal 1, 2, 3, and 4.

In step 411, the ranking of the separation values is determined depending on the magnitude of each separation value. Also, separation values which rank high are extracted by a predetermined number. In step 413, the terminal allows only elements respectively corresponding to the extracted separation values to remain among elements of the first brain wave data matrix, and computes a second covariance matrix.

In step 415, the terminal carries out the DKLT on the first brain wave data matrix obtained in step 403 by using the second covariance matrix, and computes a second brain wave data matrix having reduced data amount. The size of brain wave data is reduced through the process illustrated in FIG. 4. Accordingly, the second brain wave data matrix determined based on the procedure of FIG. 4 is employed in such a manner as to be efficiently used in a terminal which requires a relatively small amount of resources.

To help understanding, steps 401 to 415 will be exemplified. Brain wave data required to construct a database on brain waves will be referred to as source data. For convenience' sake in a description, it is assumed that the number of source data equals ‘10’, and sampling on each data is performed ten times. After sampling, the data size of each brain wave becomes ‘10.’ Source data is expressed as a matrix, and when source data having a matrix form is referred to as a first brain wave data matrix, the first brain wave data matrix is expressed as the following Equation (2):

$$source = [ \begin{matrix}Data 1 \\ Data 2 \\ \\ Data 10\end{matrix} ] , \qquad ( 2 )$$

where the size of the first brain wave data matrix is 1 by 10.

The size of a first covariance matrix on the first brain wave data matrix is 10 by 10. In addition, the DKLT is performed on the first brain wave data matrix by using the first covariance matrix. Then, separation values on all elements of the first brain wave data matrix are computed. Thereafter, two column elements best expressing such a characteristic that Data 1 is distinguished from other Data are extracted among ten column elements of Data 1. (Because the two separation values which rank high are selected among ten separation values, they have a probability of a high rank of 20%. This rate can vary.) Also, on column elements of each of the other Data, two separation values which rank high are extracted. Then, when elements respectively corresponding to the extracted separation values remain among transformed first covariance matrices, a second covariance matrix is produced, and the data size thereof is 10 by 2.

When the DKLT is performed again on the original first brain wave data matrix with a data size of 10 by 10 by using the second covariance matrix having a data size of 10 by 2, the first brain wave data matrix having a size of 10 by 2 becomes a second brain wave data matrix having a reduced data size.

The abovementioned steps can be summarized as follows. The second covariance having a reduced data amount is computed on the first brain wave data matrix (To evaluate the second covariance having a reduced data amount, separation values are computed by using the first covariance matrix.), a DKLT is performed again on the first brain wave data matrix by using the second covariance, and the second brain wave data matrix having a reduced matrix size is produced. While the second brain wave data matrix has reduced data size, it expresses a feature of each brain wave data.

When the number of brain wave data equals ‘1’, the size of a brain wave data matrix is 1 by 10, and when the brain wave data matrix passes through the procedure illustrated in FIG. 4., a produced matrix has a data size of 1 by 2. This can be applied to a step of finding a codeword on brain waves measured in step 303 illustrated in FIG. 3.

FIG. 5 is a block diagram illustrating the configuration of a terminal which operates by using brain wave data according to the present invention.

With reference to FIG. 5, an interface unit 510 receives brain wave data through the brain wave detection device 220 illustrated in FIG. 2. When brain wave data corresponds to a signal which is not digitized, sampling can be performed. Conventionally, sampling is performed every sampling time at 200 msec, but the sampling time can vary based on setting.

In FIG. 5, a data processing unit 520 reduces the size of brain wave data measured according to the procedure illustrated in FIG. 4. A data search unit 530 receives the reduced brain wave data, and searches a memory 540 for brain wave data matched to the measured brain waves. In a process for finding the brain wave data, the data search unit 530 can use a matched filter. The found brain wave data is delivered to a control unit 550 which in turn searches the memory 540 for a codeword corresponding to the brain wave data, and controls an Application (AP) 560 to operate. To this end, it is assumed that in the memory 540 illustrated in FIG. 5, multiple pieces of brain wave data respectively corresponding to multiple codewords (numerals, characters, instructions, etc.) are previously stored as a database (not shown).

Also, in FIG. 5, in the memory 540, an operating system program for an operation of the terminal, and various kinds of temporary data can be stored. In addition, a display unit 570 receives an operation state of the terminal using brain wave data from the control unit 550, and visually displays the received operation state. The AP 560 is equipped with at least one application program executed according to a control signal received from the control unit 550, and includes the functions provided to the terminal, such as a shortcut key input, text message transfer, and the like.

The merits and effects of exemplary embodiments, as disclosed in the present invention, and as so configured to operate above, will be described as follows.

With the above configuration, a user of a terminal enables an application provided from the terminal to be performed by using brain waves of the user without a direct input through a key input unit of the terminal. Hence, it is intended that there is no inconvenience for a person with disabilities who has difficulty in directly operating the terminal to operate the terminal to perform communications.

In particular, by using brain wave data having reduced size both in a brain wave database and for measured brain waves, even in a terminal having limited storage capacity, brain wave data is efficiently processed, and time necessary to recognize brain waves is reduced, thereby enabling improvement of the overall performance in a system.

While the invention has been shown and described with reference to certain exemplary embodiments thereof, it will be understood by those skilled in the art that various changes in form and details may be made therein without departing from the spirit and scope of the invention as defined by the appended claims.

What is claimed is:

1. A method for operating a terminal, the method comprising the steps of:
receiving a current brain wave of a user of the terminal;
finding an existence of data matched to the received current brain wave among a previously constructed database having reduced data sizes of brain waves of the user of the terminal and corresponding to at least one codeword; and operating the terminal according to a codeword corresponding to the brain wave matched data when the matched data is found.

2. The method as claimed in claim 1, wherein constructing the database having reduced data sizes comprises:
computing a first data matrix on the brain waves of the user of the terminal;
computing a first covariance matrix on the first data matrix;
performing a Discrete Karhunen-Lo'eve Transform (DKLT) on the first data matrix by using the first covariance matrix;
determining the number of separation values of the DKLT transformed first data matrix by a prescribed number;
extracting respective elements corresponding to the determined separation values among the first covariance matrices and determining a second covariance matrix;
performing the DKLT on the first data matrix by using the determined second covariance matrix and determining a second data matrix; and
configuring the database by using the second data matrix.

3. The method as claimed in claim 2, wherein the separation value is determined by:
$${P}_{m ; a / b}^{r} ( k ) = \frac{{[ {\overline{\tilde{z}}}_{m / a}^{r} ( k ) - {\overline{\tilde{z}}}_{m / b}^{r} ( k ) ]}^{2}}{{\sum }_{i = 1}^{I} {[ {\tilde{z}}_{m / a ; i}^{r} ( k ) - {\overline{\tilde{z}}}_{m / a}^{r} ( k ) ]}^{2} + {\sum }_{i = 1}^{I} {[ {\tilde{z}}_{m / b ; i}^{r} ( k ) - {\overline{\tilde{z}}}_{m / b}^{r} ( k ) ]}^{2}} ,$$
where ‘a’ and ‘b’ represent brain wave data different from each other, ‘m’ represents a serial number corresponding to a brain wave detection part, and ‘r’ represents the number of times brain wave data is detected.

4. The method as claimed in claim 1, wherein receiving a current brain wave comprises:
computing a first data matrix on the received current brain wave;
computing a first covariance matrix on the first data matrix;
performing a DKLT on the first data matrix by using the first covariance matrix;
determining the number of separation values of the DKLT transformed first data matrix by a prescribed number;
extracting respective elements corresponding to the determined separation values among the first covariance matrices and determining a second covariance matrix; and
performing the DKLT on the first data matrix by using the determined second covariance matrix and determining a second data matrix.

5. The method as claimed in claim 4, wherein the separation value is determined by:
$${P}_{m ; a / b}^{r} ( k ) = \frac{{[ {\overline{\tilde{z}}}_{m / a}^{r} ( k ) - {\overline{\tilde{z}}}_{m / b}^{r} ( k ) ]}^{2}}{{\sum }_{i = 1}^{I} {[ {\tilde{z}}_{m / a ; i}^{r} ( k ) - {\overline{\tilde{z}}}_{m / a}^{r} ( k ) ]}^{2} + {\sum }_{i = 1}^{I} {[ {\tilde{z}}_{m / b ; i}^{r} ( k ) - {\overline{\tilde{z}}}_{m / b}^{r} ( k ) ]}^{2}} ,$$
where ‘a’ and ‘b’ represent brain wave data different from each other, ‘m’ represents a serial number corresponding to a brain wave detection part, and ‘r’ represents the number of times brain wave data is detected.

6. An apparatus for operating a terminal, the apparatus comprising:
a memory for storing a previously constructed database having reduced data sizes of brain waves of a user of the terminal and corresponding to at least one codeword;
a data processing unit for processing a current brain wave of the user of the terminal;
a data search unit for finding data matched to the processed current brain wave among the database; and
a control unit for operating the terminal according to a codeword corresponding to the brain wave matched data when the matched data is found.

7. The apparatus as claimed in claim 6, wherein the database stored in the memory is constructed by:
computing a first data matrix on the brain waves of the user of the terminal;
computing a first covariance matrix on the first data matrix;
performing a Discrete Karhunen-Lo'eve Transform (DKLT) on the first data matrix by using the first covariance matrix;
determining the number of separation values of the DKLT transformed first data matrix by a prescribed number;
extracting respective elements corresponding to the determined separation values among the first covariance matrices and determining a second covariance matrix;
performing the DKLT on the first data matrix by using the determined second covariance matrix and determining a second data matrix; and
configuring the database by using the second data matrix.

8. The apparatus as claimed in claim 7, wherein the separation value is determined by:
$${P}_{m ; a / b}^{r} ( k ) = \frac{{[ {\overline{\tilde{z}}}_{m / a}^{r} ( k ) - {\overline{\tilde{z}}}_{m / b}^{r} ( k ) ]}^{2}}{{\sum }_{i = 1}^{I} {[ {\tilde{z}}_{m / a ; i}^{r} ( k ) - {\overline{\tilde{z}}}_{m / a}^{r} ( k ) ]}^{2} + {\sum }_{i = 1}^{I} {[ {\tilde{z}}_{m / b ; i}^{r} ( k ) - {\overline{\tilde{z}}}_{m / b}^{r} ( k ) ]}^{2}} ,$$
where ‘a’ and ‘b’ represent brain wave data different from each other, ‘m’ represents a serial number corresponding to a brain wave detection part, and ‘r’ represents the number of times brain wave data is detected.

9. The apparatus as claimed in claim 6, wherein the data processing unit carries out:
computing a first data matrix on the current brain wave of the user of the terminal;
computing a first covariance matrix on the first data matrix;
performing a DKLT on the first data matrix by using the first covariance matrix;
determining the number of separation values of the DKLT transformed first data matrix by a prescribed number;
extracting respective elements corresponding to the determined separation values among the first covariance matrices and determining a second covariance matrix;
performing the DKLT on the first data matrix by using the determined second covariance matrix and determining a second data matrix; and
reducing a size of the current brain wave by using the second data matrix.

10. The apparatus as claimed in claim 9, wherein the separation value is determined by:
$${P}_{m ; a / b}^{r} ( k ) = \frac{{[ {\overline{\tilde{z}}}_{m / a}^{r} ( k ) - {\overline{\tilde{z}}}_{m / b}^{r} ( k ) ]}^{2}}{{\sum }_{i = 1}^{I} {[ {\tilde{z}}_{m / a ; i}^{r} ( k ) - {\overline{\tilde{z}}}_{m / a}^{r} ( k ) ]}^{2} + {\sum }_{i = 1}^{I} {[ {\tilde{z}}_{m / b ; i}^{r} ( k ) - {\overline{\tilde{z}}}_{m / b}^{r} ( k ) ]}^{2}} ,$$
where ‘a’ and ‘b’ represent brain wave data different from each other, ‘m’ represents a serial number corresponding to a brain wave detection part, and ‘r’ represents the number of times brain wave data is detected.
